(19) United States
(12) Patent Application Publication
(10) Pub. No.: US 2024/0164901 A1
(43) Pub. Date: May 23, 2024

(54) HEART VALVE ATTACHMENT MECHANISM

(71) Applicant: BOSTON SCIENTIFIC SCIMED, INC., MAPLE GROVE, MN (US)

(72) Inventor: Donal Kyne, Galway (IE)

(73) Assignee: BOSTON SCIENTIFIC SCIMED, INC., MAPLE GROVE, MN (US)

(21) Appl. No.: 18/516,512

(22) Filed: Nov. 21, 2023

Related U.S. Application Data
(60) Provisional application No. 63/427,669, filed on Nov. 23, 2022.

(51) Int. Cl. A61F 2/24; A61F 2/95

(52) U.S. Cl. CPC A61F 2/2436; A61F 2/2418; A61F 2/9522; A61F 2002/9665

(57) ABSTRACT

A replacement heart valve implant includes an expandable framework configured to shift between radially collapsed and radially expanded configurations, and a plurality of valve leaflets secured to the framework. The framework includes a tubular wall defining inflow and outflow ends. The inflow end includes an attachment tab extending radially inward from the tubular wall. A replacement heart valve system includes the implant and a delivery system including a shaft assembly extending distally from a handle. A method of loading the implant includes positioning the implant adjacent an implant holding portion of the delivery system in the collapsed configuration; positioning a distal sheath of the delivery system at least partially over a groove formed in an outer surface of a stent holder of the delivery system; inserting the attachment tab into the groove; and rotating the implant relative to the delivery system.

### CROSS-REFERENCE TO RELATED APPLICATIONS

This application claims the benefit of priority of U.S. Provisional Application No. 63/427,669 filed Nov. 23, 2022, the entire disclosure of which is hereby incorporated by reference.

### TECHNICAL FIELD

The present disclosure pertains to medical devices, systems, and methods for manufacturing and/or using medical devices and/or systems. More particularly, the present disclosure pertains to features for coupling a replacement heart valve implant to an implant delivery device.

### BACKGROUND

A wide variety of intracorporeal medical devices have been developed for medical use, for example, surgical and/or intravascular use. Some of these devices include guidewires, catheters, medical device systems (e.g., for stents, grafts, replacement valves, etc.), and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

In one example, a replacement heart valve implant may comprise an expandable framework configured to shift between a radially collapsed configuration and a radially expanded configuration, and a plurality of valve leaflets secured to the expandable framework. The expandable framework may include a tubular wall defining an inflow end and an outflow end. The inflow end may include an attachment tab extending radially inward from the tubular wall.

In addition or alternatively to any example described herein, the attachment tab extends radially inward from the tubular wall between about 1.5 millimeters and about 3.5 millimeters.

In addition or alternatively to any example described herein, the attachment tab extends radially inward from the tubular wall between about 2.0 millimeters and about 3.0 millimeters.

In addition or alternatively to any example described herein, the attachment tab is axially spaced apart from the plurality of valve leaflets.

In addition or alternatively to any example described herein, the outflow end includes a plurality of stabilization arches extending axially away from the plurality of valve leaflets.

In addition or alternatively to any example described herein, the replacement heart valve implant may comprise a sealing member disposed on the expandable framework proximate the inflow end.

In addition or alternatively to any example described herein, a replacement heart valve system may comprise a replacement heart valve implant comprising an expandable framework configured to shift between a radially collapsed configuration and a radially expanded configuration, and a plurality of valve leaflets secured to the expandable framework, wherein the expandable framework includes a tubular wall defining an inflow end and an outflow end, wherein the inflow end includes an attachment tab extending radially inward from the tubular wall; and an implant delivery system comprising a handle, and an elongate shaft assembly extending distally from the handle, wherein a distal portion of the elongate shaft assembly includes an implant holding portion configured to engage with the replacement heart valve implant in the radially collapsed configuration.

In addition or alternatively to any example described herein, the elongate shaft assembly includes an outer tubular member extending distally from the handle and an inner shaft extending distally from the handle within the outer tubular member to a distal tip disposed distal of the implant holding portion.

In addition or alternatively to any example described herein, the implant holding portion may include a proximal sheath configured to cover a proximal portion of the replacement heart valve implant in the radially collapsed configuration, and a distal sheath configuration to cover a distal portion of the replacement heart valve implant in the radially collapsed configuration.

In addition or alternatively to any example described herein, the distal portion of the replacement heart valve implant includes the inflow end.

In addition or alternatively to any example described herein, the implant holding portion includes a stent holder having a groove formed in an outer surface of the stent holder, the groove being configured to receive the attachment tab.

In addition or alternatively to any example described herein, the implant holding portion includes an atraumatic transition shield disposed proximal of the stent holder and including a slot aligned with the groove of the stent holder.

In addition or alternatively to any example described herein, the groove is formed as an L-shaped slot having a first portion extending axially and a second portion extending circumferentially from the first portion.

In addition or alternatively to any example described herein, the second portion of the L-shaped slot is axially spaced apart from a proximal end of the first portion of the L-shaped slot.

In addition or alternatively to any example described herein, a method of loading a replacement heart valve implant into a replacement heart valve system may comprise: positioning a replacement heart valve implant adjacent an implant holding portion of an implant delivery system, the replacement heart valve implant having an expandable framework disposed in a radially collapsed configuration; positioning a distal sheath of the implant delivery system at least partially over a groove formed in an outer surface of a stent holder of the implant delivery system; inserting an attachment tab disposed proximate an inflow end of the expandable framework into the groove; and rotating the replacement heart valve implant relative to the implant delivery system.

In addition or alternatively to any example described herein, the groove is formed as an L-shaped slot having a first portion extending axially and a second portion extending circumferentially from the first portion.

In addition or alternatively to any example described herein, rotating the replacement heart valve implant includes translating the attachment tab into the second portion of the L-shaped slot.

In addition or alternatively to any example described herein, the method may comprise translating a proximal sheath of the implant delivery system distally over a proximal portion of the replacement heart valve implant.

In addition or alternatively to any example described herein, inserting the attachment tab into the groove includes translating the replacement heart valve implant axially relative to the implant delivery system.

In addition or alternatively to any example described herein, the attachment tab is monolithically formed with the expandable framework.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of a replacement heart valve implant;

FIG. 2 illustrates selected aspects an expandable framework associated with the replacement heart valve implant of FIG. 1;

FIG. 3 illustrates selected aspects of an implant delivery system usable with the replacement heart valve implant of FIG. 1; and

FIGS. 4-8 illustrate selected aspects related to using the implant delivery system of FIG. 3 with the replacement heart valve implant of FIG. 1.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all of the components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

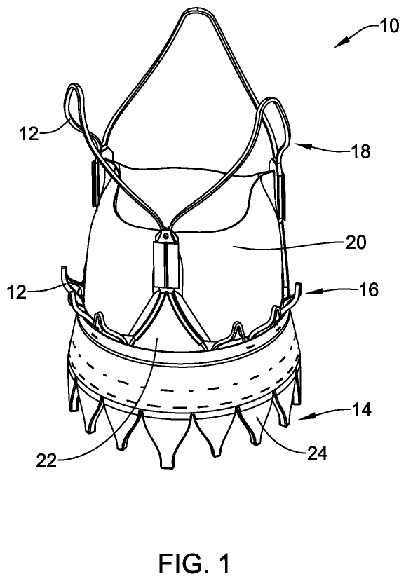

FIG. 1 illustrates selected aspects of a replacement heart valve implant 10. It should be appreciated that the replacement heart valve implant 10 can be any type of replacement heart valve (e.g., a mitral valve, an aortic valve, etc.). In use, the replacement heart valve implant 10 may be implanted (e.g., surgically or through transcatheter delivery) in a mammalian heart. The replacement heart valve implant 10 can be configured to allow oneway flow through the replacement heart valve implant 10 from an inflow end to an outflow end.

The replacement heart valve implant 10 may include an expandable framework 12 defining a central lumen. In some embodiments, the expandable framework 12 may have a substantially circular cross-section. In some embodiments, the expandable framework 12 can have a non-circular (e.g., D-shaped, elliptical, etc.) cross-section. Some suitable but non-limiting examples of materials that may be used to form the expandable framework 12, including but not limited to metals and metal alloys, composites, ceramics, polymers, and the like, are described below. The expandable framework 12 may be configured to shift between a radially collapsed configuration and a radially expanded configuration. In some embodiments, the expandable framework 12 may be self-expanding. In some embodiments, the expandable framework 12 may be self-biased toward the radially expanded configuration. In some embodiments, the expandable framework 12 may be mechanically expandable. In some embodiments, the expandable framework 12 may be balloon expandable. Other configurations are also contemplated. In some embodiments, the expandable framework 12 may include and/or define a plurality of interstices (e.g., openings) through the expandable framework 12.

In some embodiments, the expandable framework 12 may define a lower crown 14 proximate an inflow end, an upper crown 16 proximate an outflow end, and a plurality of stabilization arches 18 extending downstream from the outflow end. In some embodiments, the lower crown 14 may be disposed at the inflow end. In some embodiments, the upper crown 16 may be disposed at the outflow end. In some embodiments, the expandable framework 12 may include a tubular wall defining the central lumen, the inflow end, the outflow end, the lower crown 14, and/or the upper crown 16. In some embodiments, the plurality of stabilization arches 18 may extend downstream of and/or away from the upper crown 16 in a direction opposite the lower crown 14. In some embodiments, the upper crown 16 may be disposed longitudinally and/or axially between the lower crown 14 and the plurality of stabilization arches 18.

In some embodiments, the replacement heart valve implant 10 may include a proximal portion and a distal portion. In some embodiments, orientation of the replacement heart valve implant 10 may be related to an implant delivery device and/or a direction of implantation relative to a target site. In some embodiments, the proximal portion may include the outflow end and/or the plurality of stabilization arches 18. In some embodiments, the proximal portion may include the upper crown 16 and/or the plurality of valve leaflets 20. In some embodiments, the distal portion may include the inflow end and/or the lower crown 14. Other configurations are also contemplated.

In some embodiments, the replacement heart valve implant 10 may include a plurality of valve leaflets 20 disposed within the central lumen. The plurality of valve leaflets 20 may be coupled, secured, and/or fixedly attached to the expandable framework 12. In some embodiments, the outflow end of the expandable framework 12 may include the plurality of stabilization arches 18 extending axially away from the plurality of valve leaflets 20 and/or from a commissure (or commissures) or an attachment point (or attachment points) of the plurality of valve leaflets 20 with the expandable framework 12.

Each of the plurality of valve leaflets 20 may include a root edge coupled to the expandable framework 12 and a free edge (e.g., a coaptation edge) movable relative to the root edge to coapt with the free edges of the other leaflets along a coaptation region. In some embodiments, the plurality of valve leaflets 20 can be integrally formed with each other, such that the plurality of valve leaflets 20 is formed as a single unitary and/or monolithic unit. In some embodiments, the plurality of valve leaflets 20 may be formed integrally with other structures such as an inner skirt 22 and/or an outer skirt 24, base structures, liners, or the like.

The plurality of valve leaflets 20 may be configured to substantially restrict fluid from flowing through the replacement heart valve implant 10 in a closed position. For example, in some embodiments, the free edges of the plurality of valve leaflets 20 may move into coaptation with one another in the closed position to substantially restrict fluid from flowing through the replacement heart valve implant 10. The free edges of the plurality of valve leaflets 20 may be moved apart from each other in an open position to permit fluid flow through the replacement heart valve implant 10. In FIG. 1, the plurality of valve leaflets 20 is shown in the open position or in a partially open position (e.g., a neutral position) that the plurality of valve leaflets 20 may move to when unbiased by fluid flow.

In some embodiments, the plurality of valve leaflets 20 may be comprised of a polymer, such as a thermoplastic polymer. In some embodiments, the plurality of valve leaflets 20 may include at least 50 percent by weight of a polymer. In some embodiments, the plurality of valve leaflets 20 may be formed from bovine pericardial or other living tissue. Other configurations and/or materials are also contemplated.

In some embodiments, the replacement heart valve implant 10 may include an inner skirt 22 disposed on and/or extending along an inner surface of the expandable framework 12. In at least some embodiments, the inner skirt 22 may be fixedly attached to the expandable framework 12. The inner skirt 22 may direct fluid, such as blood, flowing through the replacement heart valve implant 10 toward the plurality of valve leaflets 20. In at least some embodiments, the inner skirt 22 may be fixedly attached to and/or integrally formed with the plurality of valve leaflets 20. The inner skirt 22 may ensure the fluid flows through the central lumen of the replacement heart valve implant 10 and does not flow around the plurality of valve leaflets 20 when they are in the closed position.

In some embodiments, the replacement heart valve implant 10 can include an outer skirt 24 disposed on and/or extending along an outer surface of the expandable framework 12. In some embodiments, the outer skirt 24 may be disposed at and/or adjacent the lower crown 14. In some embodiments, the outer skirt 24 may be disposed between the expandable framework 12 and the vessel wall in order to prevent fluid, such as blood, flowing around the replacement heart valve implant 10 and/or the expandable framework 12 in a downstream direction. The outer skirt 24 may ensure the fluid flows through the replacement heart valve implant 10 and does not flow around the replacement heart valve implant 10, so as to ensure that the plurality of valve leaflets 20 can stop the flow of fluid when in the closed position.

In some embodiments, the inner skirt 22 may include a polymer, such as a thermoplastic polymer. In some embodiments, the inner skirt 22 may include at least 50 percent by weight of a polymer. In some embodiments, the outer skirt 24 may include a polymer, such as a thermoplastic polymer. In some embodiments, the outer skirt 24 may include at least 50 percent by weight of a polymer. In some embodiments one or more of the plurality of valve leaflets 20, the inner skirt 22, and/or the outer skirt 24 may be formed of the same polymer or polymers. In some embodiments, the polymer may be a polyurethane. In some embodiments, the inner skirt 22 and/or the outer skirt 24 may be substantially impervious to fluid. In some embodiments, the inner skirt 22 and/or the outer skirt 24 may be formed from a thin tissue (e.g., bovine pericardial, etc.). In some embodiments, the inner skirt 22 and/or the outer skirt 24 may be formed from a coated fabric material. In some embodiments, the inner skirt 22 and/or the outer skirt 24 may be formed from a nonporous and/or impermeable fabric material. Other configurations are also contemplated. Some suitable but non-limiting examples of materials that may be used to form the inner skirt 22 and/or the outer skirt 24 including but not limited to polymers, composites, and the like, are described below.

In some embodiments, the inner skirt 22 and/or the outer skirt 24 may seal one of, some of, a plurality of, or each of the plurality of interstices formed in the expandable framework 12. In at least some embodiments, sealing the interstices may be considered to prevent fluid from flowing through the interstices of the expandable framework 12. In some embodiments, the inner skirt 22 and/or the outer skirt 24 may be attached to the expandable framework 12 and/or the plurality of frame struts using one or more methods including but not limited to tying with sutures or filaments, adhesive bonding, melt bonding, embedding or over molding, welding, etc.

In some embodiments, the replacement heart valve implant 10 may include a sealing member disposed on the expandable framework 12 proximate the inflow end. In some embodiments, the sealing member may include and/or may be the inner skirt 22. In some embodiments, the sealing member may include and/or may be the outer skirt 24. In some embodiments, the sealing member may include and/or may be the inner skirt 22 and the outer skirt 24. Other configurations are also contemplated.

In some embodiments, the expandable framework 12 and/or the replacement heart valve implant 10 may have an outer extent of about 23 millimeters (mm), about 25 mm, about 27 mm, about 30 mm, etc. in an unconstrained configuration (e.g., in the radially expanded configuration). In some embodiments, the expandable framework 12 and/or the replacement heart valve implant 10 may have an outer extent of about 10 mm, about 9 mm about 8 mm, about 7 mm, about 6 mm, etc. in the radially collapsed configuration. Other configurations are also contemplated.

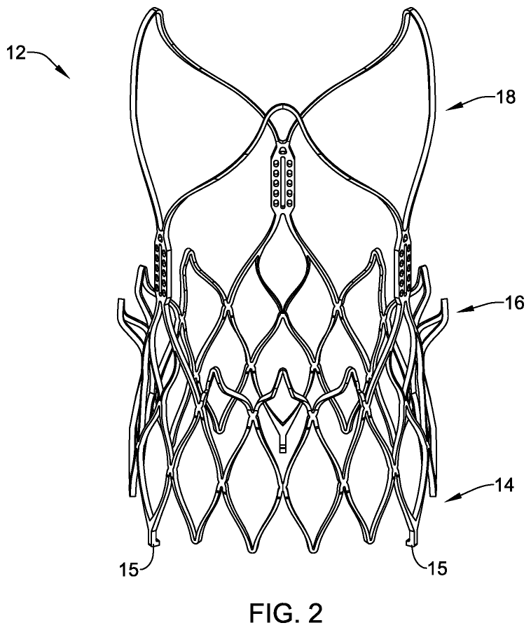

FIG. 2 illustrates selected aspects of the expandable framework 12, with other elements of the replacement heart valve implant 10 removed for clarity. In at least some embodiments, the inflow end and/or the lower crown 14 may include an attachment tab 15 extending radially inward from the expandable framework 12 and/or the tubular wall. In some embodiments, the attachment tab 15 may be formed and/or disposed at an axial extremity of the expandable framework 12. In some embodiments, the attachment tab 15 may be disposed within the distal portion of the replacement heart valve implant 10. In some embodiments, the attachment tab 15 may be disposed at a distalmost end of the expandable framework 12.

In some embodiments, the attachment tab 15 may be monolithically and/or integrally formed with the expandable framework 12. In some embodiments, the attachment tab 15 may be axially spaced apart from the plurality of valve leaflets 20. In some embodiments, the attachment tab 15 may extend radially inward from the expandable framework 12 and/or the tubular wall between about 1.5 millimeters and about 3.5 millimeters. In some embodiments, the attachment tab 15 may extend radially inward from the expandable framework 12 and/or the tubular wall between about 2.0 millimeters and about 3.0 millimeters. In some embodiments, the attachment tab 15 may extend radially inward from the expandable framework 12 and/or the tubular wall between about 2.25 millimeters and about 2.75 millimeters. In some embodiments, the attachment tab 15 may extend radially inward from the expandable framework 12 and/or the tubular wall about 2.0 millimeters, about 2.1 millimeters, about 2.2 millimeters, about 2.3 millimeters, about 2.4 millimeters, about 2.5 millimeters, about 2.6 millimeters, about 2.7 millimeters, about 2.8 millimeters, or another suitable distance. Other configurations are also contemplated.

In some embodiments, the inflow end and/or the lower crown 14 may include a plurality of attachment tabs extending radially inward from the expandable framework 12 and/or the tubular wall. In some embodiments, the plurality of attachment tabs may be spaced apart about a circumference of the expandable framework 12 and/or the tubular wall. In some embodiments, the plurality of attachment tabs may be spaced apart equidistantly about the circumference of the expandable framework 12 and/or the tubular wall.

In some embodiments, at least one attachment tab of the plurality of attachment tabs may be formed and/or disposed at an axial extremity of the expandable framework 12. In some embodiments, at least one attachment tab of the plurality of attachment tabs may be disposed within the distal portion of the replacement heart valve implant 10. In some embodiments, at least one attachment tab of the plurality of attachment tabs may be disposed at a distalmost end of the expandable framework 12. In some embodiments, at least one attachment tab of the plurality of attachment tabs may be monolithically and/or integrally formed with the expandable framework 12. In some embodiments, at least one attachment tab of the plurality of attachment tabs may be axially spaced apart from the plurality of valve leaflets 20. In some embodiments, at least one attachment tab of the plurality of attachment tabs may extend radially inward from the expandable framework 12 and/or the tubular wall between about 1.5 millimeters and about 3.5 millimeters. In some embodiments, at least one attachment tab of the plurality of attachment tabs may extend radially inward from the expandable framework 12 and/or the tubular wall between about 2.0 millimeters and about 3.0 millimeters. In some embodiments, at least one attachment tab of the plurality of attachment tabs may extend radially inward from the expandable framework 12 and/or the tubular wall between about 2.25 millimeters and about 2.75 millimeters. In some embodiments, at least one attachment tab of the plurality of attachment tabs may extend radially inward from the expandable framework 12 and/or the tubular wall about 2.0 millimeters, about 2.1 millimeters, about 2.2 millimeters, about 2.3 millimeters, about 2.4 millimeters, about 2.5 millimeters, about 2.6 millimeters, about 2.7 millimeters, about 2.8 millimeters, or another suitable distance. Other configurations are also contemplated.

In some embodiments, each attachment tab of the plurality of attachment tabs may be formed and/or disposed at an axial extremity of the expandable framework 12. In some embodiments, each attachment tab of the plurality of attachment tabs may be disposed within the distal portion of the replacement heart valve implant 10. In some embodiments, each attachment tab of the plurality of attachment tabs may be disposed at a distalmost end of the expandable framework 12. In some embodiments, each attachment tab of the plurality of attachment tabs may be monolithically and/or integrally formed with the expandable framework 12. In some embodiments, each attachment tab of the plurality of attachment tabs may be axially spaced apart from the plurality of valve leaflets 20. In some embodiments, each attachment tab of the plurality of attachment tabs may extend radially inward from the expandable framework 12 and/or the tubular wall between about 1.5 millimeters and about 3.5 millimeters. In some embodiments, each attachment tab of the plurality of attachment tabs may extend radially inward from the expandable framework 12 and/or the tubular wall between about 2.0 millimeters and about 3.0 millimeters. In some embodiments, each attachment tab of the plurality of attachment tabs may extend radially inward from the expandable framework 12 and/or the tubular wall between about 2.25 millimeters and about 2.75 millimeters. In some embodiments, each attachment tab of the plurality of attachment tabs may extend radially inward from the expandable framework 12 and/or the tubular wall about 2.0 millimeters, about 2.1 millimeters, about 2.2 millimeters, about 2.3 millimeters, about 2.4 millimeters, about 2.5 millimeters, about 2.6 millimeters, about 2.7 millimeters, about 2.8 millimeters, or another suitable distance. Other configurations are also contemplated.

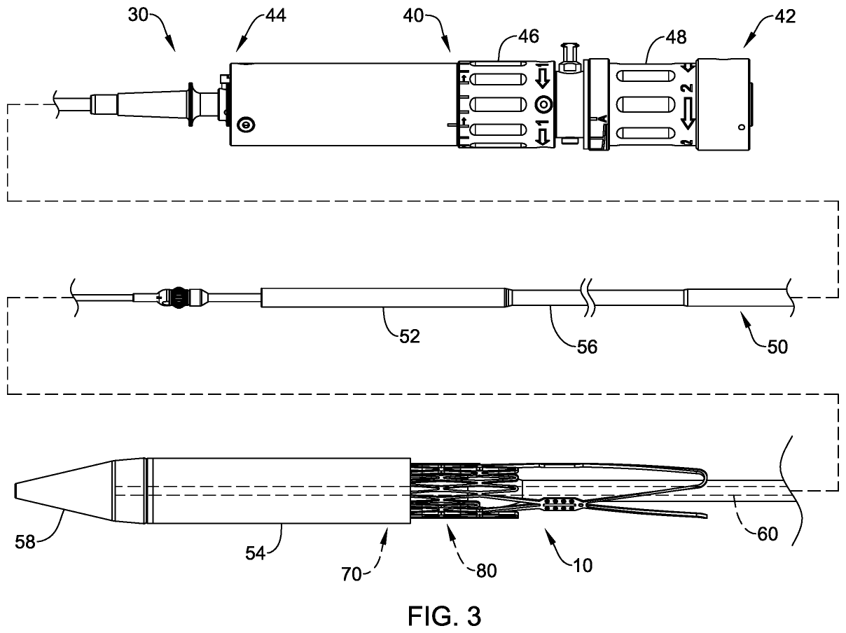

FIG. 3 illustrates selected aspects of a replacement heart valve system including an implant delivery system 30 compatible with and/or usable with the replacement heart valve implant 10. It should be noted that FIG. 3 includes at least one change of scale (e.g., all parts of the figure are not drawn to the same scale) to improve viewability and show additional detail of selected aspects of the implant delivery system 30. Additionally, some elements of the replacement heart valve implant 10 are not shown to improve clarity.

The implant delivery system 30 may include a handle 40 and an elongate shaft assembly 50 extending distally from the handle 40. The handle 40 may include a first end 42 and a second end 44 opposite the first end 42. The elongate shaft assembly 50 may extend distally from the second end 44 of the handle 40. The handle 40 may include one or more rotatable knobs. In some embodiments, the one or more rotatable knobs may include a first rotatable knob 46 and a second rotatable knob 48. In at least some embodiments, the first rotatable knob 46 and/or the second rotatable knob 48 may be configured to rotate about a central longitudinal axis of the implant delivery system 30 and/or the handle 40.

In some embodiments, a distal portion of the implant delivery system 30 and/or the elongate shaft assembly 50 may include an implant holding portion configured to engage with and/or retain the replacement heart valve implant 10 and/or the expandable framework 12 in the radially collapsed configuration. The elongate shaft assembly 50 may include an outer tubular member 56 extending distally from the handle 40 and an inner shaft 60 extending distally from the handle 40 within the outer tubular member 56 to a distal tip 58 disposed distal of the implant holding portion. In some embodiments, the implant holding portion may include a proximal sheath 52 and a distal sheath 54. In some embodiments, the inner shaft 60 may be slidably disposed within a lumen of the outer tubular member 56. In some embodiments, the inner shaft 60 may be fixedly attached to the distal sheath 54 and/or the distal tip 58. In some embodiments, the distal sheath 54 may be fixedly attached to the distal tip 58. In some embodiments, the distal sheath 54 may extend proximally from the distal tip 58. In some embodiments, the inner shaft 60 may include and/or at least partially define a guidewire lumen extending therethrough. In some embodiments, the guidewire lumen may extend through the handle 40.

In some embodiments, the handle 40 may be configured to manipulate and/or translate the proximal sheath 52 and/or the distal sheath 54 relative to each other. In some embodiments, the first rotatable knob 46 and/or the second rotatable knob 48 may be configured to manipulate and/or axially translate the proximal sheath 52 and/or the distal sheath 54 relative to each other. In some embodiments, the handle 40 may be configured to manipulate and/or translate the inner shaft 60 relative to the elongate shaft assembly 50 and/or the proximal sheath 52. In some embodiments, the first rotatable knob 46 and/or the second rotatable knob 48 may be configured to manipulate and/or axially translate the inner shaft 60 relative to the outer tubular member 56 and/or the proximal sheath 52.

During delivery of the replacement heart valve implant 10 to a treatment site, the replacement heart valve implant 10 may be disposed at least partially within the proximal sheath 52 and/or the distal sheath 54 in the radially collapsed configuration. In some embodiments, the proximal sheath 52 and/or the distal sheath 54 may collectively define the implant holding portion of the implant delivery system 30. In some embodiments, the implant holding portion may be configured to constrain the replacement heart valve implant 10 in the radially collapsed configuration. In some embodiments, the replacement heart valve implant 10 may be releasably coupled to the inner shaft 60.

In some embodiments, the proximal sheath 52 may be configured to cover the proximal portion of the replacement heart valve implant 10 in the radially collapsed configuration. As discussed herein, the proximal portion of the replacement heart valve implant 10 may include the inflow end and/or the attachment tab 15. In some embodiments, the distal sheath 54 may be configured to cover the distal portion of the replacement heart valve implant 10 in the radially collapsed configuration.

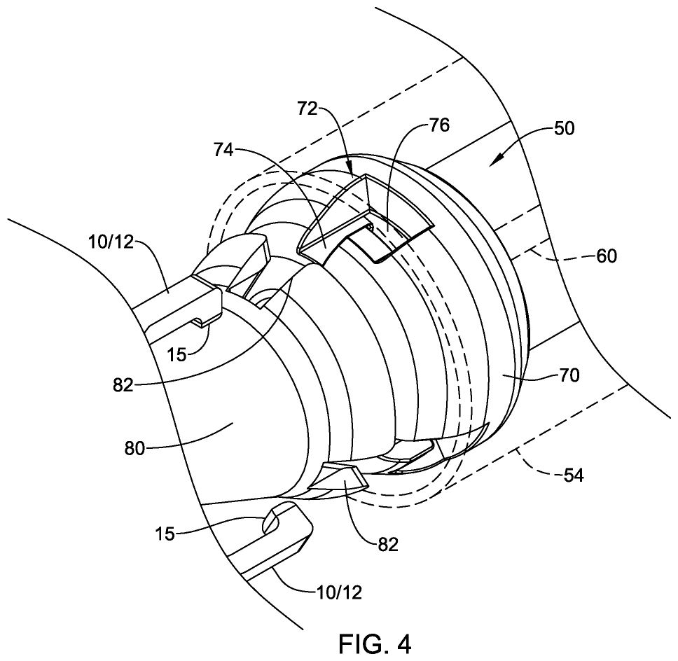

In some embodiments, the implant holding portion may include a stent holder 70 (e.g., FIGS. 4-7). In at least some embodiments, the stent holder 70 may be fixedly attached to the elongate shaft assembly 50 or a component thereof. In some embodiments, the stent holder 70 may have a generally bulbous shape. In some embodiments, the stent holder 70 may include a groove 72 formed in an outer surface of the stent holder 70. The groove 72 may be configured to receive the attachment tab 15 therein. In some embodiments, the groove 72 may be configured to slidably receive the attachment tab 15 therein.

In some embodiments, the groove 72 may be formed as an L-shaped slot having a first portion 74 extending axially and/or generally parallel to a central longitudinal axis of the elongate shaft assembly 50, and a second portion 76 extending circumferentially about the central longitudinal axis of the elongate shaft assembly 50 and/or circumferentially from the first portion 74 of the L-shaped slot.

In some embodiments, an outermost radial extent of the stent holder 70 may be disposed proximate a distal end of the stent holder 70. In some embodiments, the stent holder 70 may be tapered radially inward in a proximal direction from the outermost radial extent of the stent holder 70. In some embodiments, the first portion 74 of the L-shaped slot may extend distally from a proximal surface of the stent holder 70. In some embodiments, the first portion 74 of the L-shaped slot may extend distally from a proximally facing radially inward tapered surface of the stent holder 70. The second portion 76 of the L-shaped slot may be axially spaced apart from the proximal surface of the stent holder 70 and/or from a proximal end of the first portion 74 of the L-shaped slot.

In some embodiments, the implant holding portion may include an atraumatic transition shield 80 (e.g., FIGS. 4-7). The atraumatic transition shield 80 may be disposed adjacent the stent holder 70. In some embodiments, the atraumatic transition shield 80 may be disposed between the stent holder 70 and the handle 40. In some embodiments, the atraumatic transition shield 80 may be disposed proximal the stent holder 70. In some embodiments, the atraumatic transition shield 80 may be disposed at and/or adjacent a proximal end of the stent holder 70.

In some embodiments, the atraumatic transition shield 80 may axially overlap a portion of the stent holder 70. In some embodiments, the atraumatic transition shield 80 may be disposed radially outward of at least a portion of the stent holder 70. In some embodiments, the atraumatic transition shield 80 may be tapered radially inward in the proximal direction and/or toward the handle 40. The atraumatic transition shield 80 may be configured to prevent the replacement heart valve implant 10, the expandable framework 12, the plurality of valve leaflets 20, etc. from catching on the stent holder 70 as the implant delivery system 30 is withdrawn after deploying the replacement heart valve implant 10.

In some embodiments, the atraumatic transition shield 80 may include a slot 82 aligned with the groove 72 of the stent holder 70. In some embodiments, the slot 82 may be axially aligned with the groove 72 of the stent holder 70. In some embodiments, the slot 82 may be circumferentially aligned with the groove 72 of the stent holder 70. In some embodiments, the atraumatic transition shield 80 may include a deflectable end disposed proximate the stent holder 70. In some embodiments, a distal end of the atraumatic transition shield 80 may be resiliently flexible and/or resiliently deflectable when impinged upon by a radially inward force. If and/or when the radially inward force is removed, the atraumatic transition shield 80 may revert to its original shape. In some embodiments, the slot 82 may provide a tactile interface for and/or a tactile response when inserting the attachment tab 15 of the expandable framework 12 into the groove 72 of the stent holder 70.

In at least some embodiments, the stent holder 70 and/or the atraumatic transition shield 80 may be axially fixed relative to the handle 40. The inner shaft 60 of the elongate shaft assembly 50 and/or the implant delivery system 30 may be slidably disposed within and/or through the stent holder 70 and/or the atraumatic transition shield 80. As such, the inner shaft 60 may be slidable relative to the stent holder 70 and/or the atraumatic transition shield 80. In some embodiments, the elongate shaft assembly 50 may include at least one tubular member disposed between the inner shaft 60 and the outer tubular member 56 (e.g., over the inner shaft 60 and within the outer tubular member 56). In some embodiments, the at least one tubular member may include a plurality of layers secured together. In some embodiments, the at least one tubular member may be a plurality of tubular members that are movable (e.g., axially, rotationally, etc.) relative to each other. In at least some embodiments, the inner shaft 60 may extend through and/or may be axially slidable relative to the at least one tubular member. In some embodiments, the stent holder 70 and/or the atraumatic transition shield 80 may be secured to and/or may be fixedly attached to at least one tubular member of the elongate shaft assembly 50 that the inner shaft 60 extends and/or passes through. Other configurations are also contemplated.

As discussed herein, in some embodiments, the inflow end and/or the lower crown 14 may include a plurality of attachment tabs extending radially inward from the expandable framework 12 and/or the tubular wall. Similarly, in some embodiments, the stent holder 70 may include a plurality of grooves and the atraumatic transition shield 80 may include a plurality of slots aligned with the plurality of grooves of the stent holder 70. The plurality of grooves may be configured to receive the plurality of attachment tabs therein.

In some embodiments, the stent holder 70 may include an equal number of grooves formed therein as attachment tabs included in the replacement heart valve implant 10 and/or the expandable framework 12. In some embodiments, the stent holder 70 may include more grooves formed therein than attachment tabs included in the replacement heart valve implant 10 and/or the expandable framework 12. In some embodiments, the atraumatic transition shield 80 may include more slots formed therein than grooves included in the stent holder 70. In some embodiments, the atraumatic transition shield 80 may include an equal number of slots formed therein as grooves included in the stent holder 70. Other configurations are also contemplated.

The configuration and/or arrangement shown in FIG. 3 may be achieved according to a method of loading the replacement heart valve implant 10 into the replacement heart valve system and/or the implant delivery system 30. Some details of the method are further illustrated in FIGS. 4-7.

The method of loading the replacement heart valve implant 10 into the replacement heart valve system and/or the implant delivery system 30 may include positioning the replacement heart valve implant 10 adjacent the implant holding portion of the implant delivery system 30. The replacement heart valve implant 10 and/or the expandable framework 12 may be disposed in the radially collapsed configuration, as seen in FIG. 3.

In at least some embodiments, the method may include positioning the distal sheath 54 of the implant delivery system 30 at least partially over the groove 72 formed in the outer surface of the stent holder 70 of the implant delivery system 30, as seen in FIG. 4. To improve clarity, the distal sheath 54 is shown in phantom to indicate it presence while directing attention to other components. Accordingly, radial insertion and/or coupling of the attachment tab 15 into the groove 72 and/or the stent holder 70 may be avoided and/or prevented by the distal sheath 54.

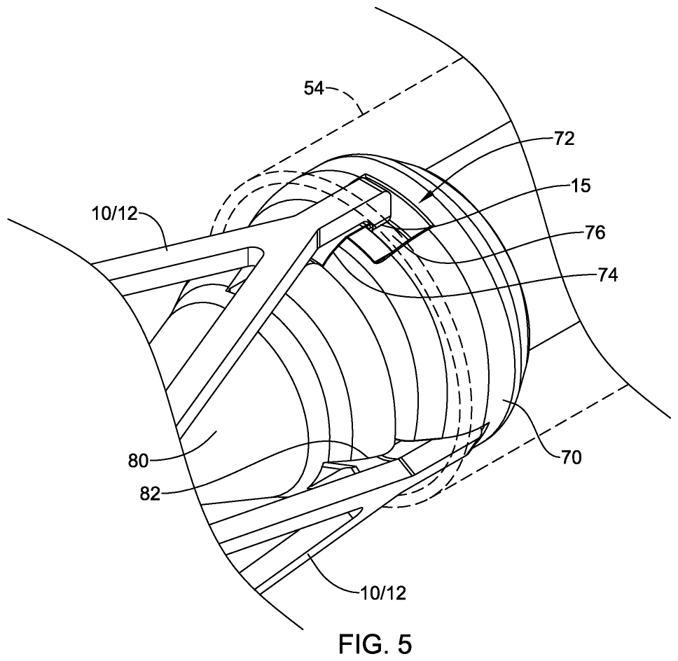

The method may include inserting the attachment tab 15 disposed proximate the inflow end of the expandable framework 12 and/or the replacement heart valve implant 10 into the groove 72 formed in the outer surface of the stent holder 70, as seen in FIG. 5. In some embodiments, inserting the attachment tab 15 into the groove 72 formed in the outer surface of the stent holder 70 may include translating the replacement heart valve implant 10, the expandable framework 12, and/or the attachment tab 15 axially relative to the implant delivery system 30 and/or the stent holder 70. In some embodiments, inserting the attachment tab 15 into the groove 72 formed in the outer surface of the stent holder 70 may include inserting and/or translating the expandable framework 12 and/or the attachment tab 15 into an interior of and/or within the distal sheath 54. In some embodiments, inserting the attachment tab 15 into the groove 72 formed in the outer surface of the stent holder 70 may include inserting and/or translating the expandable framework 12 and/or the attachment tab 15 into the interior of and/or within the distal sheath 54 without moving the distal sheath 54 relative to the stent holder 70. In some embodiments, inserting the attachment tab 15 into the groove 72 formed in the outer surface of the stent holder 70 may include inserting and/or translating the expandable framework 12 and/or the attachment tab 15 into the interior of and/or within the distal sheath 54 while holding the distal sheath 54 in a fixed position relative to the stent holder 70.

In some embodiments, inserting the attachment tab 15 into the groove 72 formed in the outer surface of the stent holder 70 may include inserting and/or translating the expandable framework 12 and/or the attachment tab 15 through the slot 82 formed in the atraumatic transition shield 80. In some embodiments, inserting the attachment tab 15 into the groove 72 formed in the outer surface of the stent holder 70 may include inserting and/or translating the expandable framework 12 and/or the attachment tab 15 through the slot 82 formed in the atraumatic transition shield 80 before and/or while inserting the attachment tab 15 into the groove 72 formed in the outer surface of the stent holder 70.

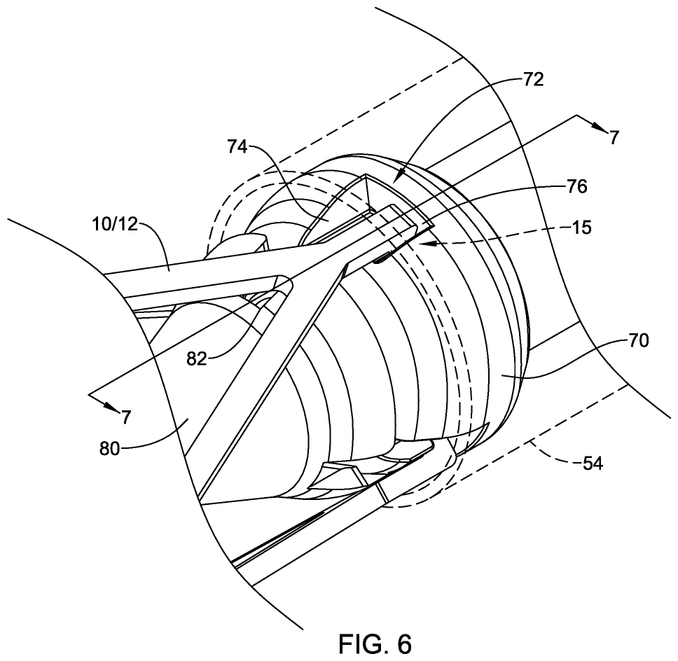

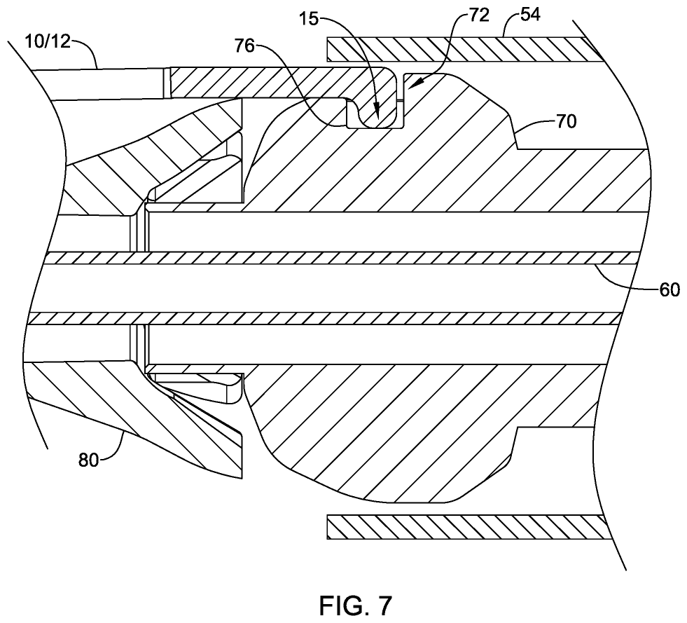

In some embodiments, the method may include rotating the replacement heart valve implant 10, the expandable framework 12, and/or the attachment tab 15 relative to the implant delivery system 30 and/or the stent holder 70. In some embodiments, rotating the replacement heart valve implant 10, the expandable framework 12, and/or the attachment tab 15 relative to the implant delivery system 30 and/or the stent holder 70 may include translating the attachment tab 15 into the second portion 76 of the L-shaped slot, as seen in FIG. 6. FIG. 7 is a partial cross-sectional view showing selected aspects of the configuration and/or relative positioning illustrated in FIG. 6.

After rotating the replacement heart valve implant 10, the expandable framework 12, and/or the attachment tab 15 relative to the implant delivery system 30 and/or the stent holder 70, the inflow end of the replacement heart valve implant 10 and/or the expandable framework 12 may be prevented from detaching from the stent holder 70. For example, the distal sheath 54 may substantially prevent radial expansion of the inflow end of the replacement heart valve implant 10 and/or the expandable framework 12. In another example, interference between the attachment tab 15 and the groove 72 of the stent holder 70 may substantially prevent axial movement of the replacement heart valve implant 10 and/or the expandable framework 12 relative to the stent holder 70 and/or the implant delivery system 30.

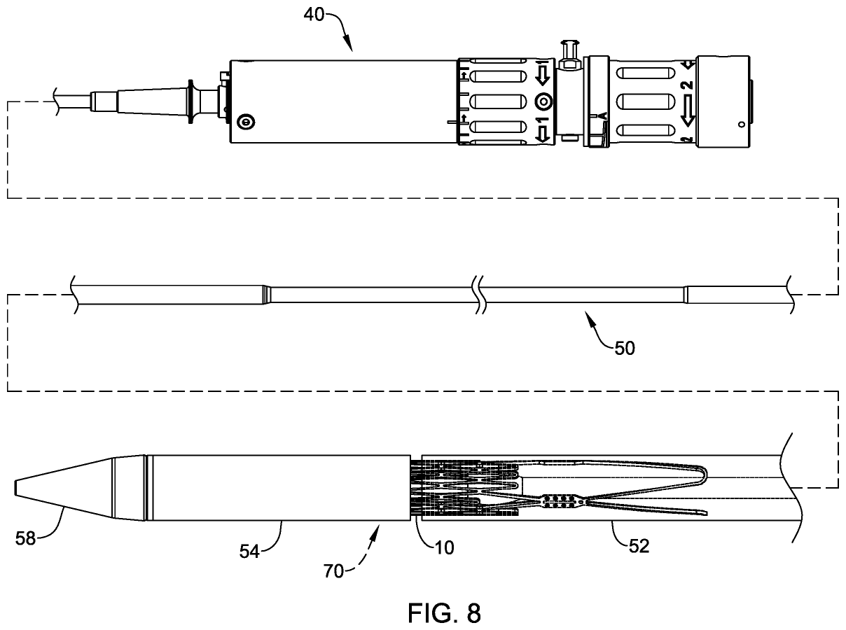

In some embodiments, the method may include translating the proximal sheath 52 of the implant delivery system 30 over the proximal portion of the replacement heart valve implant 10, as seen in FIG. 8. In some embodiments, the method may include translating the proximal sheath 52 of the implant delivery system 30 distally over the proximal portion of the replacement heart valve implant 10. In some embodiments, the method may include translating the proximal sheath 52 of the implant delivery system 30 distally relative to the inner shaft 60 and/or the stent holder 70.

In some embodiments, the replacement heart valve implant 10 and/or the expandable framework 12 may be retained in the radially collapsed configuration by the proximal sheath 52 and the distal sheath 54 in a delivery configuration of the implant delivery system 30. In some embodiments, the proximal sheath 52 may be disposed adjacent to the distal sheath 54 in the delivery configuration. In some embodiments, the proximal sheath 52 may abut the distal sheath 54 in the delivery configuration. In some embodiments, the proximal sheath 52 may be axially spaced apart from the distal sheath 54 in the delivery configuration. In some embodiments, the proximal sheath 52 may be axially spaced apart from the distal sheath 54 in the delivery configuration by less than 20% of an overall length of the replacement heart valve implant 10 and/or the expandable framework 12. In some embodiments, the proximal sheath 52 may be axially spaced apart from the distal sheath 54 in the delivery configuration by less than 15% of an overall length of the replacement heart valve implant 10 and/or the expandable framework 12. In some embodiments, the proximal sheath 52 may be axially spaced apart from the distal sheath 54 in the delivery configuration by less than 10% of an overall length of the replacement heart valve implant 10 and/or the expandable framework 12. In some embodiments, the proximal sheath 52 may be axially spaced apart from the distal sheath 54 in the delivery configuration by less than 5% of an overall length of the replacement heart valve implant 10 and/or the expandable framework 12.

The components, features, and/or methods described herein may simplify loading of the replacement heart valve implant 10 into the replacement heart valve system and/or the implant delivery system 30. Simplified loading may reduce training requirements for physicians and/or hospital staff using and/or preparing the replacement heart valve system. Simplified loading may reduce damage to the replacement heart valve system and/or components thereof. Simplified loading may reduce scrap rates and/or cost due to incorrect loading of the replacement heart valve implant 10. The components, features, and/or methods described herein may reduce cost to manufacture the replacement heart valve system compared to existing systems and/or products. Other benefits are also contemplated and/or expected.

In use, the implant delivery system 30 may be advanced percutaneously through the vasculature to a position adjacent to the treatment site. For example, the implant delivery system 30 may be advanced through the vasculature and across the aortic arch to a position adjacent to a defective native heart valve. Alternative approaches to treat a defective aortic valve and/or other heart valve(s) are also contemplated with the implant delivery system 30. After navigating the implant delivery system 30 and/or the implant holding portion to the treatment site, the proximal sheath 52 and/or the distal sheath 54 may be axially translated relative to each other to open the implant holding portion. When unconstrained by the implant holding portion, the replacement heart valve implant 10 and/or the expandable framework 12 may be configured to shift from the radially collapsed configuration to the radially expanded configuration. Shifting the replacement heart valve implant 10 and/or the expandable framework 12 toward the radially expanded configuration after axially translating the proximal sheath 52 and/or the distal sheath 54 away from each other and/or the stent holder 70 may permit the replacement heart valve implant 10 and/or the expandable framework 12 to decouple and/or detach from the implant delivery system 30 by translating the attachment tab 15 radially outward from the groove 72 of the stent holder 70. It will be appreciated that rotation of the replacement heart valve implant 10 and/or the expandable framework 12 relative to the implant delivery system 30 and/or the stent holder 70 is not required to release the replacement heart valve implant 10 and/or the expandable framework 12.

In at least some interventions, the replacement heart valve implant 10 may be deployed within the native heart valve (e.g., the native heart valve is left in place and not excised). Alternatively, the native heart valve may be removed (such as through valvuloplasty, for example) and the replacement heart valve implant 10 may be deployed in its place as a replacement. Some suitable but non-limiting materials for the implant delivery system 30, the handle 40, the elongate shaft assembly 50, the proximal sheath 52, the distal sheath 54, the inner shaft 60, the stent holder 70, the atraumatic transition shield 80, and/or components or elements thereof, for example metallic materials and/or polymeric materials, are described below.

The materials that can be used for the various components of the replacement heart valve system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the replacement heart valve implant, the expandable framework, the plurality of valve leaflets, the implant delivery system, the handle, the elongate shaft assembly, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A replacement heart valve implant, comprising:
an expandable framework configured to shift between a radially collapsed configuration and a radially expanded configuration; and
a plurality of valve leaflets secured to the expandable framework;
wherein the expandable framework includes a tubular wall defining an inflow end and an outflow end;
wherein the inflow end includes an attachment tab extending radially inward from the tubular wall.

2. The replacement heart valve implant of claim 1, wherein the attachment tab extends radially inward from the tubular wall between about 1.5 millimeters and about 3.5 millimeters.

3. The replacement heart valve implant of claim 2, wherein the attachment tab extends radially inward from the tubular wall between about 2.0 millimeters and about 3.0 millimeters.

4. The replacement heart valve implant of claim 1, wherein the attachment tab is axially spaced apart from the plurality of valve leaflets.

5. The replacement heart valve implant of claim 1, wherein the outflow end includes a plurality of stabilization arches extending axially away from the plurality of valve leaflets.

6. The replacement heart valve implant of claim 1, further comprising a sealing member disposed on the expandable framework proximate the inflow end.

7. A replacement heart valve system, comprising:
a replacement heart valve implant, comprising:
an expandable framework configured to shift between a radially collapsed configuration and a radially expanded configuration; and
a plurality of valve leaflets secured to the expandable framework;
wherein the expandable framework includes a tubular wall defining an inflow end and an outflow end;
wherein the inflow end includes an attachment tab extending radially inward from the tubular wall; and
an implant delivery system, comprising:
a handle; and
an elongate shaft assembly extending distally from the handle, wherein a distal portion of the elongate shaft assembly includes an implant holding portion configured to engage with the replacement heart valve implant in the radially collapsed configuration.

8. The replacement heart valve system of claim 7, wherein the elongate shaft assembly includes an outer tubular member extending distally from the handle and an inner shaft extending distally from the handle within the outer tubular member to a distal tip disposed distal of the implant holding portion.

9. The replacement heart valve system of claim 7, wherein the implant holding portion includes:
a proximal sheath configured to cover a proximal portion of the replacement heart valve implant in the radially collapsed configuration; and
a distal sheath configuration to cover a distal portion of the replacement heart valve implant in the radially collapsed configuration.

10. The replacement heart valve system of claim 9, wherein the distal portion of the replacement heart valve implant includes the inflow end.

11. The replacement heart valve system of claim 7, wherein the implant holding portion includes a stent holder having a groove formed in an outer surface of the stent holder, the groove being configured to receive the attachment tab.

12. The replacement heart valve system of claim 11, wherein the implant holding portion includes an atraumatic transition shield disposed proximal of the stent holder and including a slot aligned with the groove of the stent holder.

13. The replacement heart valve system of claim 11, wherein the groove is formed as an L-shaped slot having a first portion extending axially and a second portion extending circumferentially from the first portion.

14. The replacement heart valve system of claim 13, wherein the second portion of the L-shaped slot is axially spaced apart from a proximal end of the first portion of the L-shaped slot.

15. A method of loading a replacement heart valve implant into a replacement heart valve system, comprising:
positioning a replacement heart valve implant adjacent an implant holding portion of an implant delivery system, the replacement heart valve implant having an expandable framework disposed in a radially collapsed configuration;
positioning a distal sheath of the implant delivery system at least partially over a groove formed in an outer surface of a stent holder of the implant delivery system;
inserting an attachment tab disposed proximate an inflow end of the expandable framework into the groove; and
rotating the replacement heart valve implant relative to the implant delivery system.

16. The method of claim 15, wherein the groove is formed as an L-shaped slot having a first portion extending axially and a second portion extending circumferentially from the first portion.

17. The method of claim 16, wherein rotating the replacement heart valve implant includes translating the attachment tab into the second portion of the L-shaped slot.

18. The method of claim 15, further comprising translating a proximal sheath of the implant delivery system distally over a proximal portion of the replacement heart valve implant.

19. The method of claim 15, wherein inserting the attachment tab into the groove includes translating the replacement heart valve implant axially relative to the implant delivery system.

20. The method of claim 15, wherein the attachment tab is monolithically formed with the expandable framework.
